(19) United States
(12) Patent Application Publication
(10) Pub. No.: US 2016/0166836 A1
(43) Pub. Date: Jun. 16, 2016

(54) ELECTRICAL STIMULATION SYSTEM WITH OPERATING ROOM CABLE/WOUND BANDAGE AND METHODS OF MAKING AND USING

(71) Applicant: Boston Scientific Neuromodulation Corporation, Valencia, CA (US)

(72) Inventors: Christopher Shanahan, Santa Rosa, CA (US); Joshua Dale Howard, Winnetka, CA (US)

(21) Appl. No.: 14/962,938

(22) Filed: Dec. 8, 2015

Related U.S. Application Data
(60) Provisional application No. 62/091,363, filed on Dec. 12, 2014.

(51) Int. Cl. A61N 1/375; A61N 1/372

(52) U.S. Cl. CPC A61N 1/3752; A61N 1/37241

(57) ABSTRACT

An operating room cable assembly for electrically coupling at least one implantable electrical stimulation lead to a trial stimulator includes an elongated body; a trial stimulator connector disposed at one end of the elongated body; and a lead connector disposed at another end of the elongated body. The lead connector includes a connector body, a connector cover, a cover fastener disposed on the connector body, at least one lead channel to each receive a lead or lead extension, conductors coupled to the elongated body to make electrical contact with terminals disposed on the lead or lead extension, and a layer of medical adhesive disposed along a bottom surface of the lead connector. The layer of medical adhesive and the lead connector are configured and arranged for disposition over an exit wound from which the at least one lead or lead extension extends out of a skin of a patient.

### CROSS-REFERENCE TO RELATED APPLICATIONS

This application claims the benefit under 35 U.S.C. §119(e) of U.S. Provisional Patent Application Ser. No. 62/091,363 filed Dec. 12, 2014, which is incorporated herein by reference.

### FIELD

The present invention is directed to the area of implantable electrical stimulation systems and methods of making and using the systems. The present invention is also directed implantable electrical stimulation systems having an operating room cable that also acts as a wound bandage, as well as methods of making and using the cable and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is an operating room cable assembly for electrically coupling at least one implantable electrical stimulation lead to a trial stimulator. The operating room cable assembly includes an elongated body having a first end portion and an opposing second end portion; a trial stimulator connector disposed along the second end portion of the elongated body; and a lead connector disposed along the first end portion of the elongated body and electrically coupled to the trial stimulator connector. The lead connector has a top surface and a bottom surface, opposite the top surface, and is configured and arranged to mechanically receive a proximal end portion of at least one electrical stimulation lead or lead extension. The lead connector includes a connector body, a connector cover coupled to the connector body, a cover fastener disposed on the connector body to receive a portion of the connector cover and to hold the connector cover closed over the connector body, at least one lead channel to receive the at least one electrical stimulation lead or lead extension, a plurality of conductors coupled to the elongated body to make electrical contact with terminals disposed on the at least one electrical stimulation lead or lead extension when the at least one electrical stimulation lead or lead extension is received in the at least one lead channel, and a layer of medical adhesive disposed along a bottom surface of the lead connector. The layer of medical adhesive and the lead connector are configured and arranged for disposition over an exit wound from which the at least one electrical stimulation lead or lead extension extends out of a skin of a patient.

In at least some embodiments, the at least one lead channel is two lead channels, where each lead channel receives a proximal end portion of a different electrical stimulation lead or lead extension. In at least some embodiments, the lead connector includes an exterior surface with at least a portion of the exterior surface of the lead connector coated with a soft polymer material. In at least some embodiments, the lead connector further includes a water-resistant cover to be disposed over the connector cover and connector body and to permit the patient to take a shower with the lead connector covering the exit wound.

In at least some embodiments, the connector body defines at least one lead aperture through the bottom surface of the lead connector. In at least some embodiments, the lead connector further includes a sidewall disposed between the top and bottom surface of the lead connector and the lead connector defines at least one lead aperture through the sidewall of the lead connector.

In at least some embodiments, the lead connector is water-resistant when the connector cover is closed to permit the patient to take a shower with the lead connector covering the exit wound. In at least some embodiments, the cover fastener is slidable along the connector body. In at least some embodiments, the connector cover is sufficiently flexible to allow a portion of the connector cover to be folded underneath the cover fastener. In at least some embodiments, the connector cover is hingedly coupled to the connector body. In at least some embodiments, a portion of each of the lead channels is defined in both the connector body and the connector cover.

Another embodiment is an insertion kit including any of the operating room cable assemblies described above and at least one electrical stimulation lead. Each electrical stimulation lead has a distal end portion and a proximal end portion and includes electrodes disposed along the distal end portion of the electrical stimulation lead, terminals disposed along the proximal end portion of the electrical stimulation lead, and conductors electrically coupling the electrodes to the terminals. The proximal end portion of the electrical stimulation lead is insertable into the lead connector of the operating room cable assembly.

In at least some embodiments, the insertion kit also includes at least one lead extension. Each lead extension has a distal end portion and a proximal end portion and includes a connector disposed on the distal end portion to receive the proximal end portion of the electrical stimulation lead, terminals disposed along the proximal end portion of the lead extension, and conductors electrically coupling the connector to the terminals of the lead extension. The proximal end portion of the lead extension is insertable into the lead connector of the operating room cable assembly.

Yet another embodiment is an insertion kit including any of the operating room cable assemblies described above and at least one lead extension. Each lead extension having a distal end portion and a proximal end portion and includes a connector disposed on the distal end portion to receive the proximal end portion of an electrical stimulation lead, terminals disposed along the proximal end portion of the lead extension, and conductors electrically coupling the connector to the terminals of the lead extension. The proximal end portion of the lead extension is insertable into the lead connector of the operating room cable assembly.

Another embodiment is a trial stimulation arrangement for an electrical stimulation system that includes any of the insertion kits described above and a trial stimulator to generate electrical stimulation signals. The trial stimulator is disposed external to a patient and coupleable to the trial stimulator connector of the operating room cable assembly of the insertion kit.

A further embodiment is a method for performing a trial stimulation on a patient that includes providing any of the operating room cable assemblies described above; advancing a distal end portion of a first electrical stimulation lead into the patient with a proximal end portion of the first electrical stimulation lead extending outward from the patient, where the distal end portion of the first electrical stimulation lead is advanced to a position where electrodes disposed along the distal end portion of the first electrical stimulation lead are in proximity to a target stimulation location; placing the proximal end portion of the first electrical stimulation lead into a one of the at least one lead channel of the lead connector of the operating room cable assembly while the connector cover is in an open position; closing the connector cover with the cover fastener receiving a portion of the connector cover; and electrically coupling the trial stimulator connector of the operating room cable assembly to a trial stimulator.

Yet another embodiment is a method for performing a trial stimulation on a patient that includes providing any of the operating room cable assemblies described above; advancing a distal end portion of a first electrical stimulation lead into the patient with a proximal end portion of the first electrical stimulation lead extending outward from the patient, where the distal end portion of the first electrical stimulation lead is advanced to a position where electrodes disposed along the distal end portion of the first electrical stimulation lead are in proximity to a target stimulation location; coupling the proximal end portion of the first electrical stimulation lead to a connector of a lead extension; placing a proximal end portion of the lead extension into a one of the at least one lead channel of the lead connector of the operating room cable assembly while the connector cover is in an open position; closing the connector cover with the cover fastener receiving a portion of the connector cover; and electrically coupling the trial stimulator connector of the operating room cable assembly to a trial stimulator.

In at least some embodiments, any of the methods described above include placing the lead connector over an exit wound in the skin of the patient with the layer of medical adhesive adhering to the skin.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a paddle lead electrically coupled to a control module, according to the invention;

FIG. 2 is a schematic view of one embodiment of an electrical stimulation system that includes a percutaneous lead electrically coupled to a control module, according to the invention;

FIG. 3A is a schematic view of one embodiment of the control module of FIG. 1 configured and arranged to electrically couple to an elongated device, according to the invention;

FIG. 3B is a schematic view of one embodiment of a lead extension configured and arranged to electrically couple the elongated device of FIG. 2 to the control module of FIG. 1, according to the invention;

FIG. 4 is a schematic illustration of components of one embodiment of a trial stimulation system, according to the invention;

FIG. 5 is a schematic perspective view of one embodiment of a lead connector of an operating room cable assembly, according to the invention;

FIG. 6A is a schematic perspective top view of another embodiment of a lead connector of an operating room cable assembly, according to the invention;

FIG. 6B is a schematic perspective bottom view of the lead connector of FIG. 6A, according to the invention; and

FIG. 7 is a schematic perspective view of a water-resistant cover to be used with a lead connector, according to the invention.

### DETAILED DESCRIPTION

The present invention is directed to the area of implantable electrical stimulation systems and methods of making and using the systems. The present invention is also directed implantable electrical stimulation systems having an operating room cable that also acts as a wound bandage, as well as methods of making and using the cable and electrical stimulation systems.

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; and 8,364,278; and U.S. Patent Application Publication No. 2007/0150036, all of which are incorporated by reference.

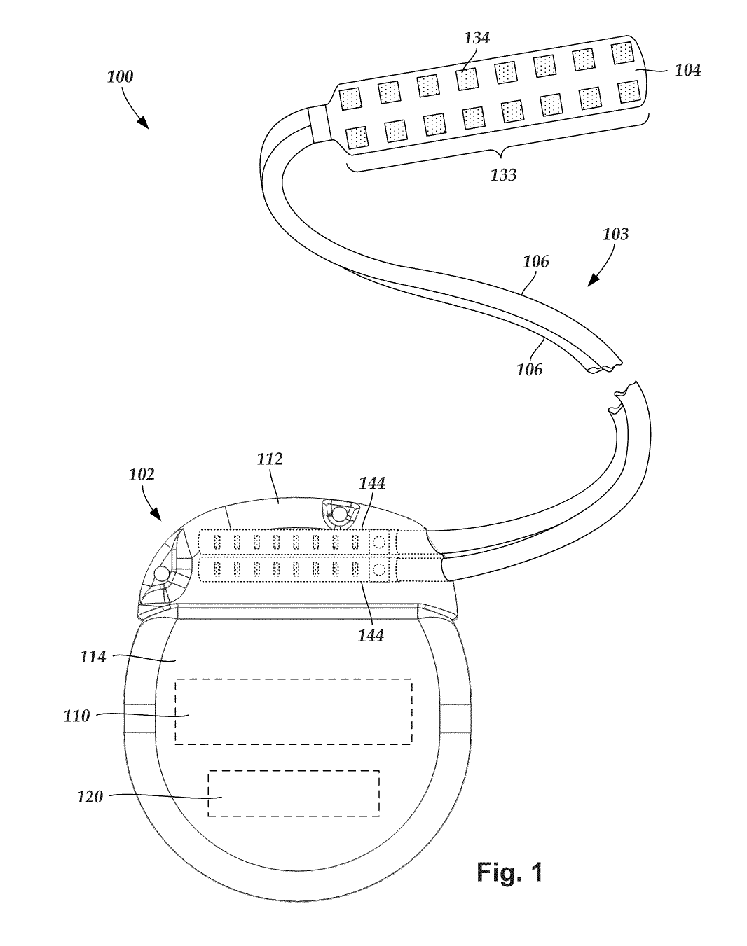

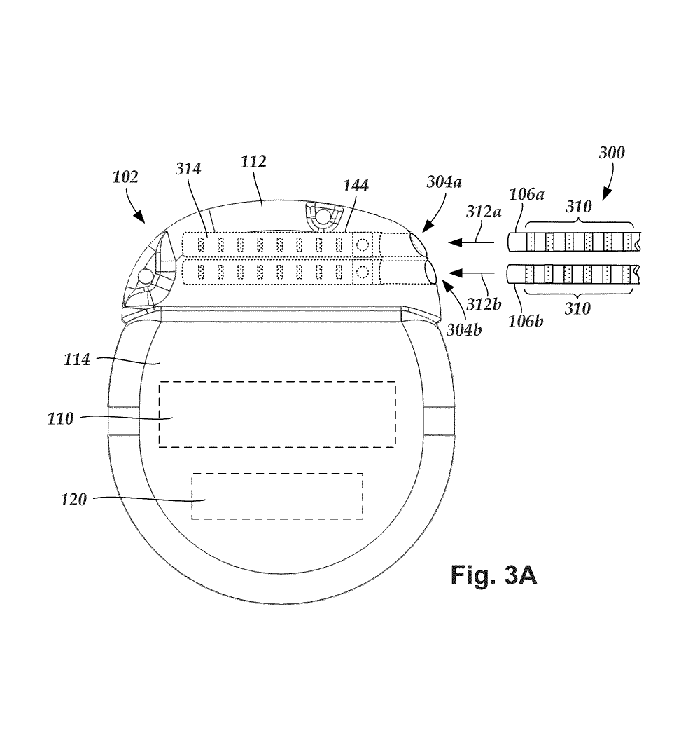

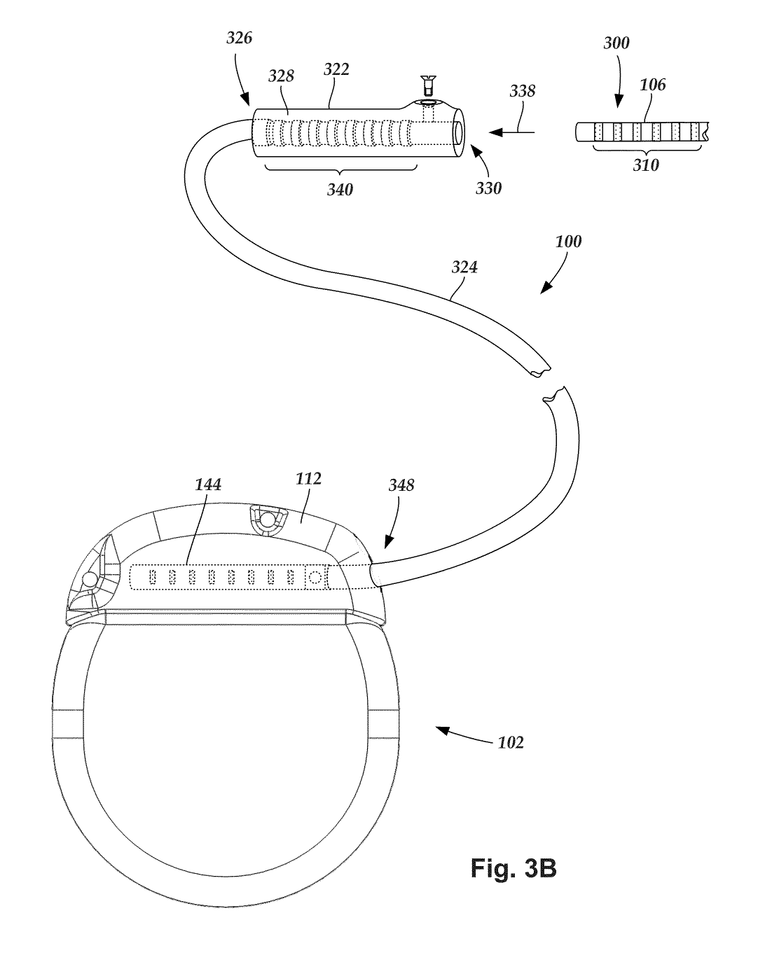

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes a paddle body 104 and one or more lead bodies 106. In FIG. 1, the lead 103 is shown having two lead bodies 106. It will be understood that the lead 103 can include any suitable number of lead bodies including, for example, one, two, three, four, five, six, seven, eight or more lead bodies 106. An array 133 of electrodes, such as electrode 134, is disposed on the paddle body 104, and an array of terminals (e.g., 310 in FIG. 3A-3B) is disposed along each of the one or more lead bodies 106.

It will be understood that the electrical stimulation system can include more, fewer, or different components and can have a variety of different configurations including those configurations disclosed in the electrical stimulation system references cited herein. For example, instead of a paddle body, the electrodes can be disposed in an array at or near the distal end of a lead body forming a percutaneous lead.

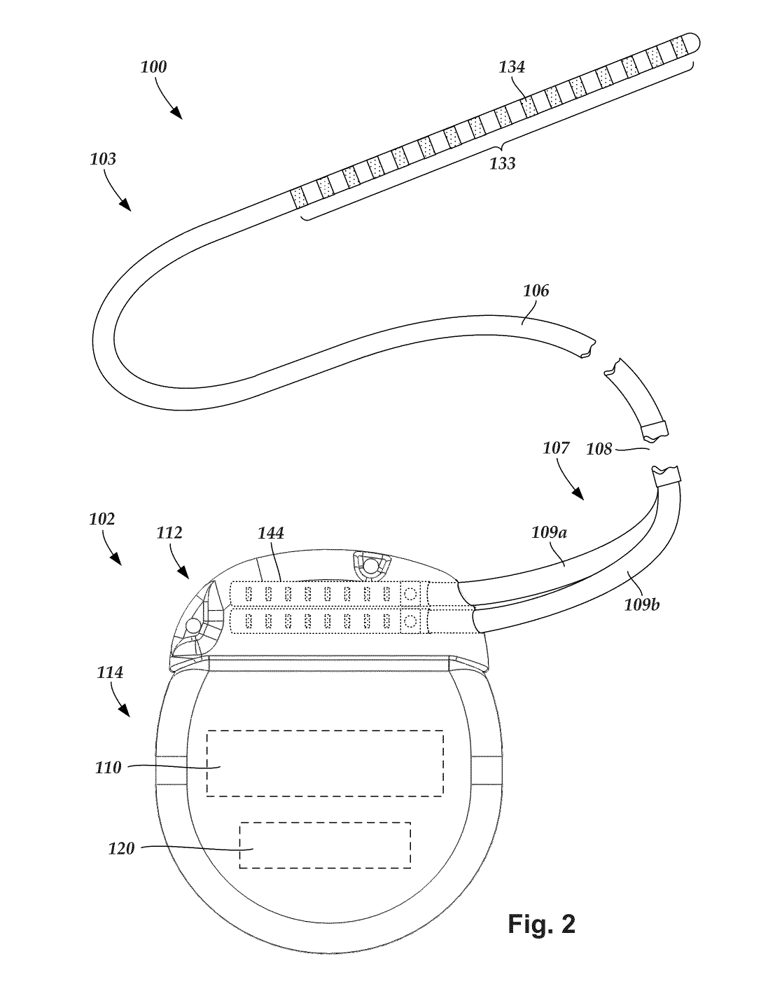

FIG. 2 illustrates schematically another embodiment of the electrical stimulation system 100, where the lead 103 is a percutaneous lead. In FIG. 2, the electrodes 134 are shown disposed along the one or more lead bodies 106. In at least some embodiments, the lead 103 is isodiametric along a longitudinal length of the lead body 106.

The lead 103 can be coupled to the control module 102 in any suitable manner. In FIG. 1, the lead 103 is shown coupling directly to the control module 102. In at least some other embodiments, the lead 103 couples to the control module 102 via one or more intermediate devices (324 in FIG. 3B). For example, in at least some embodiments one or more lead extensions 324 (see e.g., FIG. 3B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

In FIG. 2, the electrical stimulation system 100 is shown having a splitter 107 configured and arranged for facilitating coupling of the lead 103 to the control module 102. The splitter 107 includes a splitter connector 108 configured to couple to a proximal end of the lead 103, and one or more splitter tails 109a and 109b configured and arranged to couple to the control module 102 (or another splitter, a lead extension, an adaptor, or the like).

With reference to FIGS. 1 and 2, the control module 102 typically includes a connector housing 112 and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

The electrical stimulation system or components of the electrical stimulation system, including the paddle body 104, the one or more of the lead bodies 106, and the control module 102, are typically implanted into the body of a patient. The electrical stimulation system can be used for a variety of applications including, but not limited to deep brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

Any suitable number of electrodes 134 can be disposed on the lead including, for example, four, five, six, seven, eight, nine, ten, eleven, twelve, fourteen, sixteen, twenty-four, thirty-two, or more electrodes 134. In the case of paddle leads, the electrodes 134 can be disposed on the paddle body 104 in any suitable arrangement. In FIG. 1, the electrodes 134 are arranged into two columns, where each column has eight electrodes 134.

The electrodes of the paddle body 104 (or one or more lead bodies 106) are typically disposed in, or separated by, a non-conductive, biocompatible material such as, for example, silicone, polyurethane, polyetheretherketone (“PEEK”), epoxy, and the like or combinations thereof. The one or more lead bodies 106 and, if applicable, the paddle body 104 may be formed in the desired shape by any process including, for example, molding (including injection molding), casting, and the like. The non-conductive material typically extends from the distal ends of the one or more lead bodies 106 to the proximal end of each of the one or more lead bodies 106.

In the case of paddle leads, the non-conductive material typically extends from the paddle body 104 to the proximal end of each of the one or more lead bodies 106. Additionally, the non-conductive, biocompatible material of the paddle body 104 and the one or more lead bodies 106 may be the same or different. Moreover, the paddle body 104 and the one or more lead bodies 106 may be a unitary structure or can be formed as two separate structures that are permanently or detachably coupled together.

Terminals (e.g., 310 in FIGS. 3A-3B) are typically disposed along the proximal end of the one or more lead bodies 106 of the electrical stimulation system 100 (as well as any splitters, lead extensions, adaptors, or the like) for electrical connection to corresponding connector contacts (e.g., 314 in FIG. 3A). The connector contacts are disposed in connectors (e.g., 144 in FIGS. 1-3B; and 322 FIG. 3B) which, in turn, are disposed on, for example, the control module 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 3A is a schematic side view of one embodiment of a proximal end of one or more elongated devices 300 configured and arranged for coupling to one embodiment of the control module connector 144. The one or more elongated devices may include, for example, one or more of the lead bodies 106 of FIG. 1, one or more intermediate devices (e.g., a splitter, the lead extension 324 of FIG. 3B, an adaptor, or the like or combinations thereof), or a combination thereof.

The control module connector 144 defines at least one port into which a proximal end of the elongated device 300 can be inserted, as shown by directional arrows 312a and 312b. In FIG. 3A (and in other figures), the connector housing 112 is shown having two ports 304a and 304b. The connector housing 112 can define any suitable number of ports including, for example, one, two, three, four, five, six, seven, eight, or more ports.

The control module connector 144 also includes a plurality of connector contacts, such as connector contact 314, disposed within each port 304a and 304b. When the elongated device 300 is inserted into the ports 304a and 304b, the connector contacts 314 can be aligned with a plurality of terminals 310 disposed along the proximal end(s) of the elongated device(s) 300 to electrically couple the control module 102 to the electrodes (134 of FIG. 1) disposed on the paddle body 104 of the lead 103. Examples of connectors in control modules are found in, for example, U.S. Pat. Nos. 7,244,150 and 8,224,450, which are incorporated by reference.

FIG. 3B is a schematic side view of another embodiment of the electrical stimulation system 100. The electrical stimulation system 100 includes a lead extension 324 that is configured and arranged to couple one or more elongated devices 300 (e.g., one of the lead bodies 106 of FIGS. 1 and 2, the splitter 107 of FIG. 2, an adaptor, another lead extension, or the like or combinations thereof) to the control module 102. In FIG. 3B, the lead extension 324 is shown coupled to a single port 304 defined in the control module connector 144. Additionally, the lead extension 324 is shown configured and arranged to couple to a single elongated device 300. In alternate embodiments, the lead extension 324 is configured and arranged to couple to multiple ports 304 defined in the control module connector 144, or to receive multiple elongated devices 300, or both.

A lead extension connector 322 is disposed on the lead extension 324. In FIG. 3B, the lead extension connector 322 is shown disposed at a distal end 326 of the lead extension 324. The lead extension connector 322 includes a connector housing 328. The connector housing 328 defines at least one port 330 into which terminals 310 of the elongated device 300 can be inserted, as shown by directional arrow 338. The connector housing 328 also includes a plurality of connector contacts, such as connector contacts 340. When the elongated device 300 is inserted into the port 330, the connector contacts 340 disposed in the connector housing 328 can be aligned with the terminals 310 of the elongated device 300 to electrically couple the lead extension 324 to the electrodes (134 of FIGS. 1 and 2) disposed along the lead (103 in FIGS. 1 and 2).

In at least some embodiments, the proximal end of the lead extension 324 is similarly configured and arranged as a proximal end of the lead 103 (or other elongated device 300). The lead extension 324 may include a plurality of electrically conductive wires (not shown) that electrically couple the connector contacts 340 to a proximal end 348 of the lead extension 324 that is opposite to the distal end 326. In at least some embodiments, the conductive wires disposed in the lead extension 324 can be electrically coupled to a plurality of terminals (not shown) disposed along the proximal end 348 of the lead extension 324. In at least some embodiments, the proximal end 348 of the lead extension 324 is configured and arranged for insertion into a connector disposed in another lead extension (or another intermediate device). In other embodiments (and as shown in FIG. 3B), the proximal end 348 of the lead extension 324 is configured and arranged for insertion into the control module connector 144.

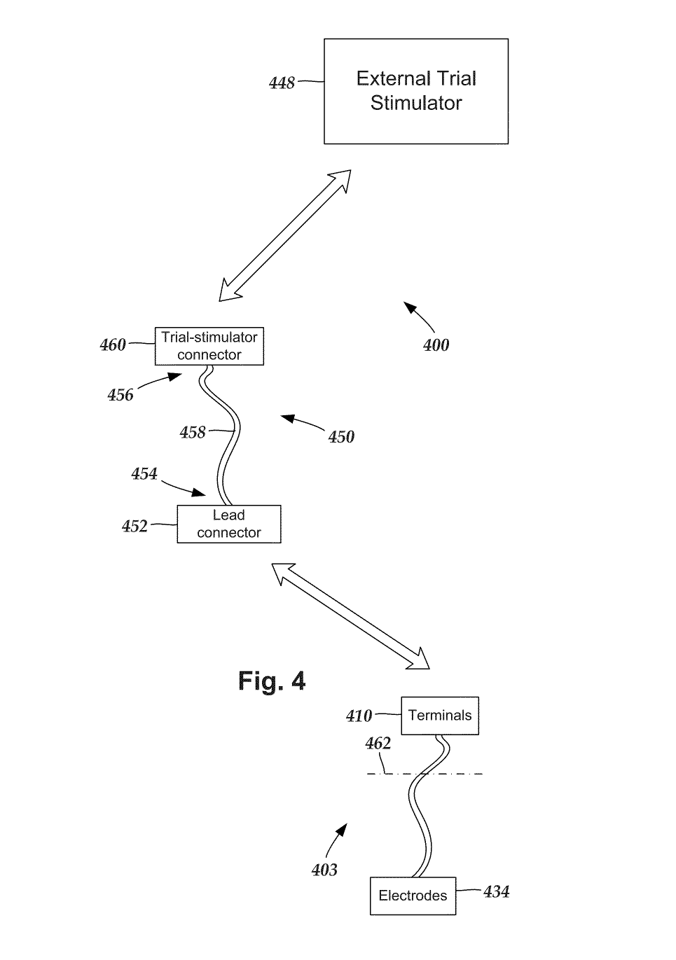

Turning to FIG. 4, during implantation of the lead into a patient it is sometimes desirable to test the positioning or functionality of the electrodes within the patient prior to completion of the implantation. One way to test electrode positioning or functionality is to implant an electrode-including distal end portion of a lead (and, optionally, one or more lead extensions) into the patient. The proximal end portion of the lead (or lead extension) can then be electrically coupled to a trial stimulator that is disposed external to the patient to perform trial stimulations using the electrodes. Once it is determined that the electrodes are properly positioned and functioning within desired parameters, the trial stimulator can be removed from the proximal end portion of the lead (or lead extension) and replaced with an implantable control module, and the implantation can be completed.

In some embodiments, the trial stimulations can continue for two, four, six, eight, twelve, or more hours or for one, two, three, four, five or more days. In these instances, the patient may be in a hospital or other care facility. In some embodiments, the trial stimulations may continue for an extended period (e.g., 2-10 days or more) where the patient is sent home with the lead, cable, and trial stimulator to assess the effectiveness of the therapy to determine if a permanent implanted system will be effective in treating the medical condition. During the trial stimulations, the lead can be electrically coupled to the trial stimulator by electrically coupling the proximal end portion of the lead (or lead extension) to an operating room cable (“cable”) which, in turn, is electrically coupled to the trial stimulator. In some cases, when multiple leads are implanted into a patient, multiple leads (or lead extensions) may be coupled to the cable.

FIG. 4 is a schematic view of one embodiment of a trial stimulation arrangement 400 that includes a lead 403, a trial stimulator 448, and an operating room cable assembly 450, that couples the lead 403 to the trial stimulator 448. The lead 403 includes an array of electrodes 434 and an array of terminals 434. The terminals 434 are configured and arranged to couple the electrodes 434 to the trial stimulator 448 when the operating room cable assembly 450 is coupled to each of the lead 403 and the trial stimulator 448.

During operation, the electrodes 434 are disposed internal to the patient, while the terminals 434 remain external to the patient, as shown in FIG. 4 by a line 462 schematically representing patient skin. Optionally, the trial stimulation arrangement 400 includes one or more additional devices (e.g., a lead extension, an operating room cable extension, a splitter, an adaptor, or the like or any combination thereof).

The operating room cable assembly 450 includes an elongated body 458 having a first end portion 454 and an opposing second end portion 456, a lead connector 452, and a trial stimulator connector 460. The lead connector 452 is disposed along the first end portion 454 of the operating room cable assembly 450 and is coupleable to the terminals 434 of the lead 403 (or lead extension). The trial stimulator connector 460 is disposed along the second end portion 456 of the operating room cable assembly 450 and is coupleable to the trial stimulator 448, either directly or via one or more operating room cable extensions.

Conventionally, the lead connectors of the operating room cable assembly are relatively bulky and uncomfortable for the patient. In addition, a bandage is used to cover the exit wound site where the lead or lead extension exits the skin. In contrast to conventional operating room cable assemblies, the operating room cable assemblies described below utilize the lead connector, instead of a bandage, to cover the exit wound and preferably are designed and arranged for patient comfort. In some embodiments, the lead connector can be made waterproof or sufficiently water-resistant to allow a patient to shower with concern about water entering the lead connector or exit wound area.

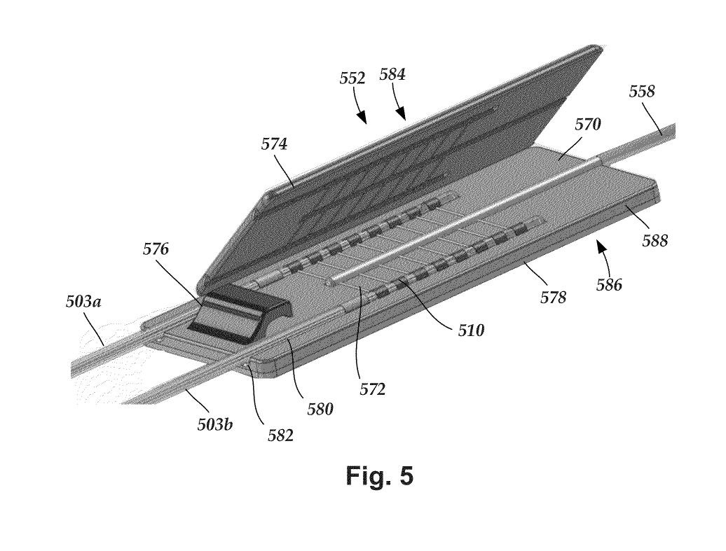

FIG. 5 illustrates one embodiment of a lead connector 552 of an operating room cable assembly (such as operating room cable assembly 450 of FIG. 4) with an elongated body 558 extending from the lead connector and, in the illustrated embodiments, the proximal ends of two leads 503a, 503b (or two lead extensions or any combination thereof) inserted into the lead connector 552. It will be recognized that the lead connector can be modified to receive any number of leads or lead extensions including, for example, one, two, three, four, or more leads or lead extensions. It will also be recognized that the lead may have two or more lead bodies (see, for example, FIG. 2) and each lead body can be inserted into the lead connector. For example, a lead with two proximal lead bodies can be coupled to the lead connector 552 with a different lead body in each lead channel 580. In the description below, leads will be referred to in connection with the lead connector. It will be understood, however, that a lead extension can be used in place of any of the leads.

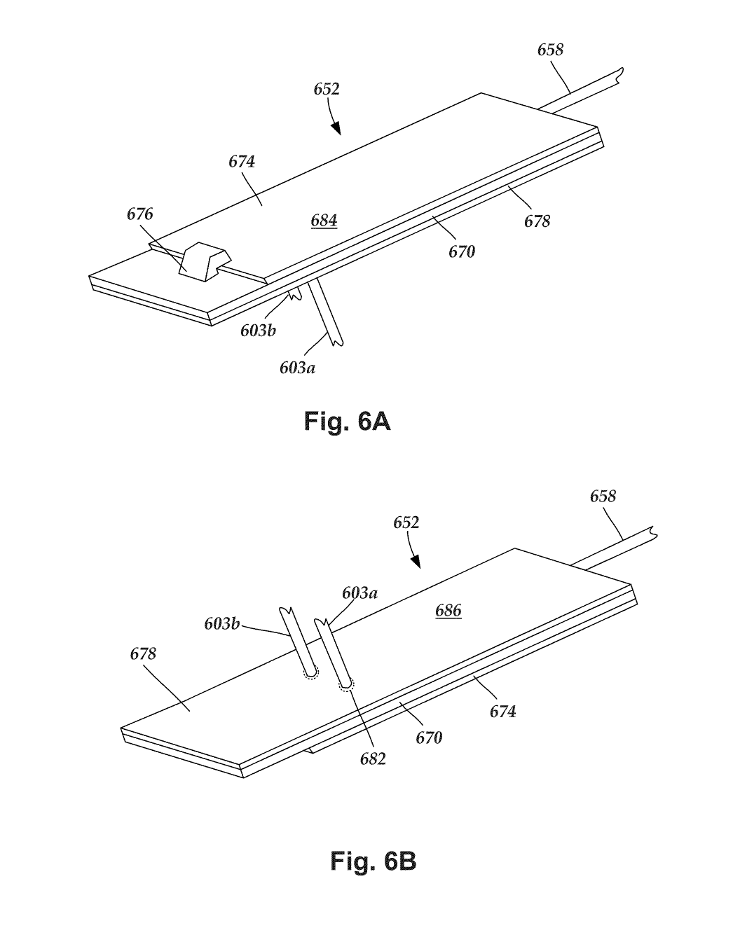

The lead connector 552 includes a connector body 570, a connector cover 574, a cover fastener 576, conductors 572, one or more lead channels 580, one or more lead apertures 582, and a medical adhesive layer 578. The connector body 570 and, optionally, the connector cover 574 define one or more lead channels 580 and one or more lead apertures 582 to receive the proximal ends of the leads 503a, 503b. The one or more lead channels 580 can be larger in diameter than the proximal ends of the leads 503a, 503b or can be the same diameter or slightly smaller in diameter than the proximal ends of the leads 503a, 503b to hold the leads within the lead channels using a frictional or interference fit. In the embodiment of FIG. 5, the leads 503a, 503b exit the lead connector 552 through one or more lead apertures 582 on a sidewall of the lead connector 552. In other embodiments, as illustrated in FIGS. 6A-6B and described in more detail below, the one or more lead apertures may be formed in a bottom surface of the connector body.

In some embodiments, one or both of the connector body 570 and connector cover 574 can be formed of a relatively flexible polymer material to be more comfortable for the patient. The connector body 570, however, should be sufficiently rigid to hold the leads 503a, 503b in electrical connection with the conductors 572. In at least some embodiments, the connector body 570 and, optionally, the connector cover 574 may be formed of a rigid polymer material that is covered (completely or, for example, over at least some or all of the portions that form the exterior of the lead connector 522) with a softer or more flexible polymer material, such as rubber, silicone, or polyurethane. Use of softer or more flexible materials can enhance patient comfort. Preferably, the connector body 570 and connector cover 574 have a relatively low profile for patient comfort.

The connector cover 574 can be hinged or otherwise attached to the connector body 570 so that the lead connector 552 can be opened to receive the leads 503a, 503b. In at least some embodiments, the cover fastener 576 may be slidable along the connector body 570 to fasten or allow opening of the connector cover 574. In other embodiments, the connector cover 574 can be sufficiently flexible to allow the cover to be bent and inserted underneath a portion of the cover fastener 576. In at least some embodiments, the cover fastener 576 is made of a relatively hard plastic or rubber sufficient to retain the cover 574 in place after fastening the cover. It will be understood that other types of cover fasteners can also be used including, but not limited to, pins, clips, sutures, and the like.

The conductors 572 are arranged in the connector body 572 (or alternatively in the connector cover 574) to make contact with the terminals 510 of the leads 503a, 503b. In at least some embodiments, the conductors 572 include wires or traces. In at least some embodiments, each of the conductors 572 may include a hemispherical or planar contact resting in, or adjacent to, the lead channels 580 to make contact with one or more terminals 510 of the leads 503a. The conductors 572 may be electrically coupled to contacts or conductors of the elongated body 558 or the conductors 572 may extend along at least a portion of the elongated body. Any other suitable arrangement for conductively coupling the terminals 510 of the lead 503a, 503b to the elongated body 558 can be used.

The lead connector 552 is configured and arranged to cover the exit wound from which the leads 503a, 503b extend from the skin of the patient. In this manner, the lead connector 552 acts to both couple the leads 503a, 503b to the trial stimulator and as a bandage/covering for the exit wound. To this end, the lead connector 552 includes a layer 578 of medical adhesive that will adhere to the skin of the patient. The adhesive and connector body 570 are made of biocompatible materials that are suitable for contact with the exit wound over a period of time extending at least one day, two days, one week, or longer.

In the embodiment of FIG. 5, the leads 503a, 503b exit the lead connector 552 from the sidewall 588 between the top surface 584 and bottom surface 586 of the lead connector. In this embodiment, the leads will then loop around to the exit wound site and the lead connector 552 can be placed over the exit wound site.

In another embodiment, illustrated in FIGS. 6A-6B, a lead connector 652 is arranged so that the leads 603a, 603b exit the bottom surface 686 of the lead connector, as illustrate in FIG. 6B. The lead connector 652 includes a connector body 670, a connector cover 674, a cover fastener 676, connectors (not shown), one or more lead channels (not shown), one or more lead apertures 682, a medical adhesive layer 678, and a top surface 682. The description and design considerations for each of the named elements described above for the embodiment of FIG. 5 is also applicable to the embodiment of FIGS. 6A and 6B unless indicated otherwise.

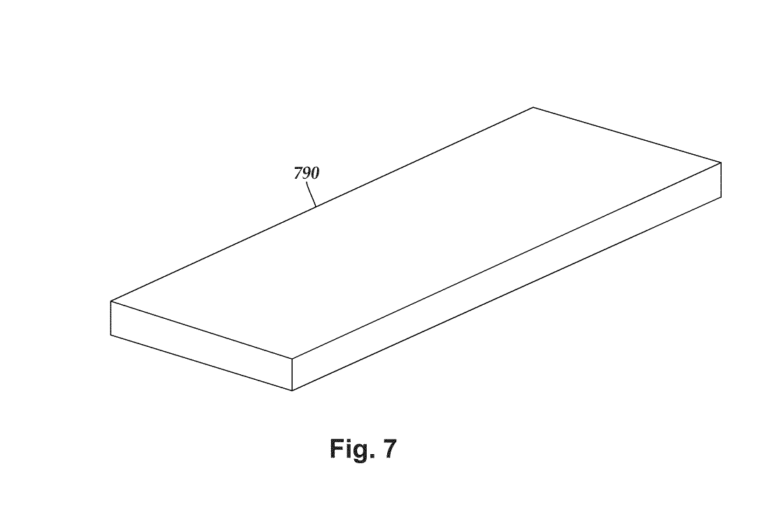

In at least some embodiments, the lead connector 562, 662 also includes a water-resistant cover 790, illustrated in FIG. 7, that fits over the other elements of the lead connector after the lead connector is placed on the skin of the patient. In at least some embodiments, the water-resistant cover 790, when properly placed over the lead connector 562, 662, can be waterproof or water-resistant sufficient for the patient to take a shower.

The water-resistant cover 790 can have a defined form, as illustrated in FIG. 7, or may be in the form of skin or other arrangement that fits snugly over the other elements of the lead connector 562, 662. The water-resistant cover 790 can include an aperture that allows exit of the elongated body 558, 658. In some embodiments, such as that illustrated in FIG. 5, the water-resistant cover can include one or more apertures that allow exit of the leads 503a, 503b.

In other embodiments, the lead connector 562, 662 itself may be made of waterproof or water-resistant materials and with a structure that resists or prevents the entry of water between the connector body 570, 670 and connector cover 574, 674 or between the layer 578, 678 of medical adhesive and the skin of the patient or any combination thereof. Making the lead connector waterproof or water-resistant (with or without a water-resistant cover 790) can be convenient for the patient and allow the patient to shower or otherwise come into contact with water without taking additional precautions.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An operating room cable assembly for electrically coupling at least one implantable electrical stimulation lead to a trial stimulator, the operating room cable assembly comprising:
an elongated body having a first end portion and an opposing second end portion;
a trial stimulator connector disposed along the second end portion of the elongated body; and
a lead connector disposed along the first end portion of the elongated body and electrically coupled to the trial stimulator connector, the lead connector having a top surface and a bottom surface, opposite the top surface, and configured and arranged to mechanically receive a proximal end portion of at least one electrical stimulation lead or lead extension, the lead connector comprising
a connector body,
a connector cover coupled to the connector body,
a cover fastener disposed on the connector body and configured and arranged to receive a portion of the connector cover and to hold the connector cover closed over the connector body,
at least one lead channel configured and arranged to receive the at least one electrical stimulation lead or lead extension,
a plurality of conductors coupled to the elongated body and configured and arranged to make electrical contact with terminals disposed on the at least one electrical stimulation lead or lead extension when the at least one electrical stimulation lead or lead extension is received in the at least one lead channel, and
a layer of medical adhesive disposed along a bottom surface of the lead connector, wherein the layer of medical adhesive and the lead connector are configured and arranged for disposition over an exit wound from which the at least one electrical stimulation lead or lead extension extends out of a skin of a patient.

2. The operating room cable assembly of claim 1, wherein the at least one lead channel is two lead channels, wherein each lead channel is configured and arranged to receive a proximal end portion of a different electrical stimulation lead or lead extension.

3. The operating room cable assembly of claim 1, wherein the lead connector comprises an exterior surface with at least a portion of the exterior surface of the lead connector coated with a soft polymer material.

4. The operating room cable assembly of claim 1, wherein the lead connector further comprises a water-resistant cover configured and arranged to be disposed over the connector cover and connector body and to permit the patient to take a shower with the lead connector covering the exit wound.

5. The operating room cable assembly of claim 1, wherein the connector body defines at least one lead aperture through the bottom surface of the lead connector.

6. The operating room cable assembly of claim 1, wherein the lead connector further comprises a sidewall disposed between the top and bottom surface of the lead connector, wherein the lead connector defines at least one lead aperture through the sidewall of the lead connector.

7. The operating room cable assembly of claim 1, wherein the lead connector is water-resistant when the connector cover is closed to permit the patient to take a shower with the lead connector covering the exit wound.

8. The operating room cable assembly of claim 1, wherein the cover fastener is slidable along the connector body.

9. The operating room cable assembly of claim 1, wherein the connector cover is sufficiently flexible to allow a portion of the connector cover to be folded underneath the cover fastener.

10. The operating room cable assembly of claim 1, wherein the connector cover is hingedly coupled to the connector body.

11. The operating room cable assembly of claim 1, wherein a portion of each of the lead channels is defined in both the connector body and the connector cover.

12. An insertion kit comprising:
the operating room cable assembly of claim 1; and
at least one electrical stimulation lead, each electrical stimulation lead having a distal end portion and a proximal end portion and comprising
a plurality of electrodes disposed along the distal end portion of the electrical stimulation lead,
a plurality of terminals disposed along the proximal end portion of the electrical stimulation lead, and
a plurality of conductors coupling the plurality of electrodes to the plurality of terminals,
wherein the proximal end portion of the electrical stimulation lead is insertable into the lead connector of the operating room cable assembly.

13. The insertion kit of claim 12, further comprising at least one lead extension, each lead extension having a distal end portion and a proximal end portion and comprising a connector disposed on the distal end portion to receive the proximal end portion of the electrical stimulation lead, a plurality of terminals disposed along the proximal end portion of the lead extension, and a plurality of conductors electrically coupling the connector to the plurality of terminals of the lead extension, wherein the proximal end portion of the lead extension is insertable into the lead connector of the operating room cable assembly.

14. An insertion kit comprising:
the operating room cable assembly of claim 1; and
at least one lead extension, each lead extension having a distal end portion and a proximal end portion and comprising a connector disposed on the distal end portion to receive the proximal end portion of an electrical stimulation lead, a plurality of terminals disposed along the proximal end portion of the lead extension, and a plurality of conductors electrically coupling the connector to the plurality of terminals of the lead extension, wherein the proximal end portion of the lead extension is insertable into the lead connector of the operating room cable assembly.

15. A trial stimulation arrangement for an electrical stimulation system, the trial stimulation arrangement comprising:
the insertion kit of claim 12; and
a trial stimulator configured and arranged to generate electrical stimulation signals, the trial stimulator disposed external to a patient and coupleable to the trial stimulator connector of the operating room cable assembly of the insertion kit.

16. A trial stimulation arrangement for an electrical stimulation system, the trial stimulation arrangement comprising:
the insertion kit of claim 14; and
a trial stimulator configured and arranged to generate electrical stimulation signals, the trial stimulator disposed external to a patient and coupleable to the trial stimulator connector of the operating room cable assembly of the insertion kit.

17. A method for performing a trial stimulation on a patient, the method comprising:
providing the operating room cable assembly of claim 1;
advancing a distal end portion of a first electrical stimulation lead into the patient with a proximal end portion of the first electrical stimulation lead extending outward from the patient, wherein the distal end portion of the first electrical stimulation lead is advanced to a position where a plurality of electrodes disposed along the distal end portion of the first electrical stimulation lead are in proximity to a target stimulation location;
placing the proximal end portion of the first electrical stimulation lead into a one of the at least one lead channel of the lead connector of the operating room cable assembly while the connector cover is in an open position;
closing the connector cover with the cover fastener receiving a portion of the connector cover; and
electrically coupling the trial stimulator connector of the operating room cable assembly to a trial stimulator.

18. The method of claim 17, further comprising placing the lead connector over an exit wound in the skin of the patient with the layer of medical adhesive adhering to the skin.

19. A method for performing a trial stimulation on a patient, the method comprising:
providing the operating room cable assembly of claim 1;
advancing a distal end portion of a first electrical stimulation lead into the patient with a proximal end portion of the first electrical stimulation lead extending outward from the patient, wherein the distal end portion of the first electrical stimulation lead is advanced to a position where a plurality of electrodes disposed along the distal end portion of the first electrical stimulation lead are in proximity to a target stimulation location;
coupling the proximal end portion of the first electrical stimulation lead to a connector of a lead extension;
placing a proximal end portion of the lead extension into a one of the at least one lead channel of the lead connector of the operating room cable assembly while the connector cover is in an open position;
closing the connector cover with the cover fastener receiving a portion of the connector cover; and
electrically coupling the trial stimulator connector of the operating room cable assembly to a trial stimulator.

20. The method of claim 19, further comprising placing the lead connector over an exit wound in the skin of the patient with the layer of medical adhesive adhering to the skin.
